(19) United States
(12) Patent Application Publication
(10) Pub. No.: US 2022/0277822 A1
(43) Pub. Date: Sep. 1, 2022

(54) PHARMACEUTICAL INJECTION ASSESSMENT SYSTEM AND PHARMACEUTICAL INJECTION ASSESSMENT PROGRAM

(71) Applicant: PHC Holdings Corporation, Tokyo (JP)

(72) Inventors: Noriyoshi TERASHIMA, Ehime (JP); Masataka NADAOKA, Ehime (JP)

(21) Appl. No.: 17/627,310

(22) PCT Filed: Sep. 3, 2020
(86) PCT No.: PCT/JP2020/033473

§ 371 Date: Jan. 14, 2022

(30) Foreign Application Priority Data
Sep. 18, 2019 (JP) 2019-169466

(51) Int. Cl. G16H 20/17; A61M 5/172; G16H 10/60

(52) U.S. Cl. CPC G16H 20/17; A61M 5/1723; G16H 10/60; A61M 2205/6009; A61M 2205/502; A61M 2205/3379; A61M 2205/581; A61M 2205/587; A61M 2230/201; A61M 2205/3313

(57) ABSTRACT

A pharmaceutical injection assessment system comprises a blood glucose level measurement device, a smartphone, and a terminal device. The blood glucose level measurement device measures the blood glucose level and acquires a dose of insulin on the basis of the measured blood glucose level. The smartphone captures a confirmation image showing the type and dose of insulin. The terminal device has a communication unit for receiving a confirmation image, and a display for prompting the administrator to confirm the type and dose of insulin.

### TECHNICAL FIELD

The present invention relates, for example, to a pharmaceutical injection assessment system and a pharmaceutical injection assessment program for determining the suitability of the type, dose, etc., of a specific pharmaceutical to be administered to a patient on the basis of measurement results for blood glucose level or other such biometric information.

### BACKGROUND ART

In recent years, in order to prevent medical errors related to the type and dose of a pharmaceutical when administering a pharmaceutical such as an intravenous drip to a patient, a medical practice determination system has been used to determine whether or not the administration is a legitimate medical practice.

For example, Patent Literature 1 discloses a medical practice determination system capable of determining whether or not the relation between a nurse, a patient, and a pharmaceutical is as it should be when a nurse administers a pharmaceutical to a patient.

### CITATION LIST

### Patent Literature

Patent Literature 1: JP-A 2005-62994 (Japanese Patent No. 4,355,851)

### SUMMARY

### Technical Problem

However, the above-mentioned conventional medical practice determination system has the following problem.

With the medical practice determination system disclosed in the above publication, when a nurse administers a pharmaceutical to a patient, specific information that identifies the nurse, the patient, and the pharmaceutical is acquired, and this identification information about nurse, patient, and pharmaceutical is referred to along with the patient-pharmaceutical table and a pharmaceutical-nurse table in order to determine whether or not the specified pharmaceutical is the proper pharmaceutical that is permitted to be administered to the specified patient. Also, the specified nurse determines whether or not he or she is the proper nurse who is permitted to administer the specified pharmaceutical, and determines whether or not the nurse, patient, and pharmaceutical are all in their proper relationship according to the results of these determinations.

Therefore, the above-mentioned medical practice determination system, no consideration whatsoever is given to determining whether or not the type and dose of a specific pharmaceutical such as insulin that should be administered promptly are appropriate, on the basis of the result of measuring biometric information such as blood glucose level in real time.

It is an object of the present invention to provide a pharmaceutical injection assessment system and a pharmaceutical injection assessment program with which the administrator is prompted to confirm the type and dose of a specific pharmaceutical set on the basis of a biometric information measurement result, thereby ensuring the administration of the specific pharmaceutical as dictated by the biometric information measurement result.

### Solution to Problem

The pharmaceutical injection assessment system according to the first invention is a pharmaceutical injection assessment system with which an administrator performs confirmation work related to the administration of a specific pharmaceutical on the basis of a measurement result for biometric information measured by the dosing person or the patient, the system comprising a biometric information measurement device, a pharmaceutical dose acquisition unit, and a terminal device. The biometric information measurement device measures biometric information. The pharmaceutical dose acquisition unit acquires the type and dose of a specific pharmaceutical on the basis of biometric information measured by the biometric information measuring device. The terminal device has a receiving unit for receiving information related to the type and dose of the specific pharmaceutical that will actually be administered to the patient, and a display unit for displaying a display prompting the administrator to confirm the type and dose of the specific pharmaceutical.

Here, in order to have the administrator determine whether or not the type and dose of a specific pharmaceutical that is set on the basis of a biometric information measurement result and that will actually be administered to the patient are appropriate, information about the type and dosage of the specific pharmaceutical is transmitted to the terminal device on the administrator side, and a display prompting confirmation is displayed on the display unit of the terminal device.

Here, blood glucose level is an example of the biometric information measured by the dosing person or the patient using the biometric information measurement device. Insulin is an example of the specific pharmaceutical to be administered to the patient on the basis of the biometric information measurement result.

Also, the pharmaceutical dose acquisition unit is provided inside the biometric information measurement device, and may acquire the dose by automatically calculating the type and dose of the pharmaceutical on the basis of the biometric information measurement result, or may acquire the dose by using the communication function of the biometric information measurement device to transmit the biometric information measurement result to the outside, and receiving or manually inputting the externally calculated dose.

Information about the type and dose of a specific pharmaceutical that will actually be administered to a patient includes, for example, confirmation images, specific pharmaceutical type and dose data, etc., captured using the camera function built into a smartphone, mobile phone, tablet terminal, or biometric information measurement device belonging to the dosing person.

The terminal device is, for example, a smartphone, mobile phone, tablet terminal, PC (personal computer), biometric information measurement device, or the like belonging to an administrator, and can be anything capable for communicating with a biometric information measurement device or between biometric information measurement devices.

Here, the dosing person who administers a specific pharmaceutical, and the administrator who confirms the type and dose of the pharmaceutical before administration, may be fixed to one or more specific people, or, for example, may be one or more people selected as needed from among a number of people on the basis of work shifts and the like. Also, the dosing person may be the administrator, and conversely the administrator may be the dosing person.

Consequently, on the administrator side, the type and dose of the pharmaceutical that will actually be administered to the patient are displayed on the basis of the information about the type and dose of the specific pharmaceutical received at the terminal device (such as a smartphone, a tablet terminal, a PC, or a biometric information measurement device), and the user can refer to the biometric information measurement result to determine suitability. Instructions from the doctor (the prescription determined by the doctor according to the patient's condition) is preferably displayed along with the pharmaceutical.

As a result, the administrator can be prompted to confirm the type and dose of the specific pharmaceutical set on the basis of the biometric information measurement result, and once this determination result by the administrator is obtained, the dosing person can reliably carry out the administration of the specific pharmaceutical as dictated by the biometric information measurement result.

The pharmaceutical injection assessment system according to the second invention is the pharmaceutical injection assessment system according to the first invention, further comprising an imaging unit that captures a confirmation image showing the type and dose of the specific pharmaceutical that will actually be administered to the patient.

Here, a confirmation image (including, for example, the label part of an insulin pharmaceutical bottle) showing the type, dose, and so forth of the specific pharmaceutical that will actually be administered to the patient is used as the information about the type and dose of a specific pharmaceutical that will actually be administered to the patient.

Here, the confirmation image can be, for example, an image including the graduated portion of a pharmaceutical injection device such as a syringe containing the specific pharmaceutical actually to be administered to a patient.

Also, the confirmation image showing the state before the insulin or other pharmaceutical is administered is captured at a timing such as immediately before the pharmaceutical injection, immediately before the pharmaceutical injection button is pressed, or immediately before the pharmaceutical injection button is operated.

Consequently, the administrator can easily determine whether or not the type and dose of the specific pharmaceutical that will actually be administered to the patient are appropriate by checking the confirmation image sent from the dosing person.

The pharmaceutical injection assessment system according to the third invention is the pharmaceutical injection assessment system according to the second invention, wherein the confirmation image includes a graduated portion showing the dose of the specific pharmaceutical in a state in which the specific pharmaceutical has been placed in a pharmaceutical injection device that administers the specific pharmaceutical to a patient.

Here, an image including a graduated portion showing the dose of the pharmaceutical injection device in the state in which the specific pharmaceutical will actually be administered to the patient is used as the confirmation image.

Consequently, the administrator can look at the graduated portion showing the dose of the pharmaceutical injection device included in the confirmation image received by the terminal device, and determine whether or not the dose of the specific pharmaceutical is appropriate for the patient whose biometric information has been measured.

The pharmaceutical injection assessment system according to the fourth invention is the pharmaceutical injection assessment system according to the second or third invention, wherein the confirmation image includes information about the patient to whom the specific pharmaceutical is to be administered.

Here, an image including information about the patient whose biometric information has been measured (such as the patient's face, patient ID, etc.) is used as the confirmation image.

Consequently, the administrator can look at the patient information included in the confirmation image received by the terminal device, and determine whether or not the type and dose of the specific pharmaceutical are appropriate for the patient whose biometric information has been measured.

The pharmaceutical injection assessment system according to the fifth invention is the pharmaceutical injection assessment system according to any of the first to fourth inventions, wherein the biometric information measurement device has a measurement unit for measuring the biometric information, and a first communication unit for transmitting the measurement result from the measurement unit.

Here, biometric information such as a blood glucose level, for example, is measured by the measurement unit, and the measurement result is transmitted from the first communication unit.

Consequently, the biometric information measured by the measurement unit can be transmitted to a component for calculating the pharmaceutical dose, which is either inside or outside the biometric information measurement device.

The pharmaceutical injection assessment system according to the sixth invention is the pharmaceutical injection assessment system according to any of the second to fourth inventions, wherein the biometric information measurement device further has the pharmaceutical dose acquisition unit and the imaging unit.

Here, the pharmaceutical dose acquisition unit provided to the biometric information measurement device acquires the type and dose of the specific pharmaceutical according to the biometric information measurement result, and the imaging unit provided to the biometric information measurement device is used to capture a confirmation image showing this dose.

Consequently, the type and dose of the specific pharmaceutical can be acquired on the basis of the biometric information measurement result obtained in the biometric information measurement device, and a confirmation image can also be captured by using the biometric information measurement device.

The pharmaceutical injection assessment system according to the seventh invention is the pharmaceutical injection assessment system according to the fifth invention, further comprising an imaging unit for capturing a confirmation image showing the type and dose of the specific pharmaceutical that will actually be administered to the patient. The first communication unit transmits the confirmation image captured by the imaging unit.

Here, in the biometric information measurement device, the first communication unit transmits the confirmation image captured by the imaging unit to a terminal device on the administrator side, such as a smartphone, a mobile phone, a tablet terminal, a PC, or the like.

Consequently, the biometric information measurement device can promptly transmit the captured confirmation image to the terminal device on the administrator side.

The pharmaceutical injection assessment system according to the eighth invention is the pharmaceutical injection assessment system according to any of the first to seventh inventions, wherein the pharmaceutical dose acquisition unit calculates the type and dose of a specific pharmaceutical on the basis of the biometric information measured by the biometric information measurement device.

Here, in the biometric information measurement device, the type and dose of a specific pharmaceutical are calculated on the basis of the measured biometric information.

Consequently, the type and dose of a specific pharmaceutical can be acquired within the biometric information measurement device.

The pharmaceutical injection assessment system according to the ninth invention is the pharmaceutical injection assessment system according to the fifth invention, wherein the pharmaceutical dose acquisition unit receives the type and dose of the specific pharmaceutical calculated on the basis of the biometric information transmitted from the first communication unit of the biometric information measurement device.

Here, the biometric information measured by the biometric information measurement device is transmitted to a pharmaceutical dose acquisition unit provided externally, and the type and dose of the specific pharmaceutical are acquired by receiving the type and dose of the specific pharmaceutical that was calculated.

Consequently, a dose that is appropriate according to the measurement result can be obtained, for example, by transmitting the biometric information measurement result to the pharmaceutical dose acquisition unit provided to an external PC or the like.

The pharmaceutical injection assessment system according to the tenth invention is the pharmaceutical injection assessment system according to any of the first to ninth inventions, wherein the terminal device causes the display unit to display at least one set of text information from among the biometric information measurement result, the type and dose of the specific pharmaceutical, the patient ID, and the dosing person ID.

Here, at least one set of text information from among the biometric information measurement result, the type and dose of the specific pharmaceutical, the patient ID, and the dosing person ID are displayed at the terminal device on the administrator side.

Consequently, the administrator can look at the display of the terminal device and determine, for example, whether or not the type and dose of the specific pharmaceutical are appropriate for the patient, whether or not the dosing person is the right one, and so forth.

The pharmaceutical injection assessment system according to the eleventh invention is the pharmaceutical injection assessment system according to any of the first to tenth inventions, wherein the terminal device further has an output unit for outputting voice information or light that prompts the administrator to take care regarding the type and dose of the specific pharmaceutical.

Here, when the administrator makes a determination regarding the administration of the specific pharmaceutical, the user is notified by voice information or light that a confirmation image has been received, for example.

Consequently, the administrator can promptly recognize that a confirmation image has been received on a smartphone, mobile phone, tablet terminal, PC, etc., so that the administrator can promptly make a determination regarding the administration of the specific pharmaceutical and can reply to the dosing person.

The pharmaceutical injection assessment system according to the twelfth invention is the pharmaceutical injection assessment system according to any of the first to eleventh inventions, wherein the terminal device further has an input unit to which the result of determining the suitability of the type and dose of the specific pharmaceutical is inputted by the administrator who has confirmed the type and dose of the specific pharmaceutical.

Here, the administrator inputs a determination result regarding the administration of the specific pharmaceutical at the terminal device on the administrator side.

Consequently, the dosing person can receive the determination regarding the administration of the specific pharmaceutical by the administrator, and take an action according to the determination result.

The pharmaceutical injection assessment system according to the thirteenth invention is the pharmaceutical injection assessment system according to any of the second to fourth inventions, wherein the terminal device further has a second communication unit that sends the dosing person a message prompting one of the following: the result of the suitability determination made by the administrator who confirmed the type and dose of the specific pharmaceutical, a retake of the confirmation image, a re-measurement of the biometric information, and a recalculation of the dose of the specific pharmaceutical, or a message indicating that the specific pharmaceutical cannot be administered.

Here, as a result of the administrator's determination regarding the administration of the specific pharmaceutical, it is possible to transmit a message prompting one of the following: confirmation of the suitability of administering the specific pharmaceutical as it is, the re-capture of a confirmation image, the re-measurement of biometric information, and the recalculation of the does of the specific pharmaceutical, or a message indicating that the specific pharmaceutical cannot be administered, for example.

Consequently, the dosing person can refer to the transmitted message based on the determination result on the administrator side, and then take a suitable action.

The pharmaceutical injection assessment system according to the fourteenth invention is the pharmaceutical injection assessment system according to any of the first to thirteenth inventions, further comprising a memory unit for storing information about employees corresponding to the administrator and the dosing person.

Here, employees, including the dosing person who administers the specific pharmaceutical to the patient, and the administrator who makes a determination regarding the suitability of this administration, are stored in the memory unit in advance.

Consequently, the dosing person and the administrator can be appropriately managed by limiting the dosing person who administers the specific pharmaceutical to the patient and the administrator who manages this administration to specific people.

The pharmaceutical injection assessment system according to the fifteenth invention is the pharmaceutical injection assessment system according to the fourteenth invention, wherein the memory unit further stores the work shifts for the employees.

Here, the work shifts for employees are stored ahead of time in the memory unit.

Consequently, in selecting an employee, the work shifts stored in the memory unit can be used to set a combination of a dosing person and an administrator from among the people who are currently working.

The pharmaceutical injection assessment system according to the sixteenth invention is the pharmaceutical injection assessment system according to the fifteenth invention, further comprising a setting unit for setting a combination of the dosing person and the administrator on the basis of the work shifts for the employees stored in the memory unit.

Here, in the setting unit, a combination of a dosing person and an administrator is automatically set from among the people on duty by using the work shifts stored in the memory unit.

Consequently, after the biometric information is measured by the biometric information measurement device, the recipient to which the confirmation image will be sent from the dosing person is automatically set, so a determination regarding the administration of a specific pharmaceutical can be made more efficiently.

The pharmaceutical injection assessment system according to the seventeenth invention is the pharmaceutical injection assessment system according to the sixteenth invention, wherein the combination of the dosing person and the administrator set in the setting unit is shared between the biometric information measurement device and the terminal device.

Here, in the biometric information measurement device on the dosing person side and the terminal device on the administrator side, preset dosing person and administrator combination information is shared by using mutual communication functions and the like.

Consequently, when the dosing person reads his or her own ID or the like into the biometric information measurement device in the course of using the biometric information measurement device, for example, the relationship with the preset administrator is automatically recognized right away.

Consequently, the dosing person can promptly recognize who the administrator is set to that day, and can have a determination made regarding the suitability of administering the specific pharmaceutical.

The pharmaceutical injection assessment system according to the eighteenth invention is the pharmaceutical injection assessment system according to the fourteenth invention, wherein the biometric information measurement device further has an information reading unit for reading a patient's personal information. The memory unit stores a setting range related to the type and dose of the specific pharmaceutical corresponding to the patient's personal information read by the information reading unit.

Here, when the patient's ID is read using an information reading unit such as a barcode reader, for example, instructions from the doctor regarding the type of the specific pharmaceutical and the upper and dose limits set for each patient stored in the memory unit are confirmed by the administrator and the dosing person.

This makes it possible to determine whether or not the type and dose of the specific pharmaceutical are appropriate, taking into consideration the instructions from the doctor. As an example, if the insulin dose calculated on the basis of the biometric information measurement result is different from the doctor's instructions, “contact the attending physician” can be displayed on the screen and an alert can be issued. This allows the dosing person or the like who has seen the alert to contact the attending physician and optimize the insulin dose calculated on the basis of a biometric information measurement result that differs from the instructions.

The pharmaceutical injection assessment system according to the nineteenth invention is the pharmaceutical injection assessment system according to any of the first to eighteenth inventions, wherein the biometric information measurement device measures blood glucose level as the biometric information, and the pharmaceutical dose acquisition unit acquires a dose of insulin as the specific pharmaceutical.

Here, blood glucose level is used as the biometric information measured by the biometric information measurement device, and insulin is used as the specific pharmaceutical to be administered to the patient.

Consequently, the proper dose of insulin is acquired on the basis of the measurement result for the blood glucose level of the patient, and the administrator can determine whether or not an appropriate amount of insulin is about to be administered with respect to the blood glucose level, by referring to a confirmation image or the like.

The pharmaceutical injection assessment system according to the twentieth invention is the pharmaceutical injection assessment system according to the second invention, wherein the imaging unit is provided to a smartphone, a mobile phone, a tablet terminal, or a PC belonging to the dosing person.

Here, a camera function installed in a smartphone, a mobile phone, a tablet terminal, or a PC belonging to the dosing person is used as the imaging unit for capturing a confirmation image.

Consequently, after measuring the biometric information, the dosing person can capture a confirmation image using the camera function of his or her smartphone or the like, and send this to the terminal device on the administrator side by using the communication function of the smartphone or the like.

The pharmaceutical injection assessment program according to the twenty-first invention is a pharmaceutical injection assessment program with which an administrator performs confirmation work related to the administration of a specific pharmaceutical on the basis of a biometric information measurement result measured by the dosing person or the patient, wherein the program causes a computer to execute a pharmaceutical injection determination method comprising the steps of: measuring the biometric information; acquiring the type and dose of the specific pharmaceutical on the basis of the measured biometric information; and receiving information about the type and dose of the specific pharmaceutical actually administered to the patient and displaying a display to prompt the administrator to confirm the type and dose of the specific pharmaceutical.

Here, in order to have the administrator determine whether or not the type and dose of the specific pharmaceutical set on the basis of the biometric information measurement result are appropriate, information about the type and dosage of the specific pharmaceutical that will actually be administered to the patient is transmitted to the terminal device on the administrator side, and a display prompting confirmation is displayed on the display unit of the terminal device.

Here, the biometric information measured by the dosing person or the patient using the biometric information measurement device includes, for example, blood glucose level and the like. The specific pharmaceutical to be administered to the patient on the basis of the biometric information measurement result includes, for example, insulin and the like.

In the step of acquiring the type and dose of the specific pharmaceutical, the dose may be acquired by using software provided in the biometric information measurement device to calculate the type and dose of the pharmaceutical automatically on the basis of the biometric information measurement result, or the communication function of the biometric information measurement device may be used to transmit the biometric information measurement result to the outside, and the dose calculated by receiving or manually inputting a dose that was calculated externally.

Information about the type and dose of the specific pharmaceutical that will actually be administered to a patient includes, for example, confirmation images, specific pharmaceutical type and dose data, etc., captured using the camera function built into a smartphone, mobile phone, or tablet terminal, or biometric information measurement device belonging to the dosing person.

The terminal device is, for example, a smartphone, mobile phone, tablet terminal, PC (personal computer), biometric information measurement device, or the like belonging to an administrator, and can be anything capable for communicating with a biometric information measurement device or between biometric information measurement devices.

Here, the dosing person who administers a specific pharmaceutical, and the administrator who confirms the type and dose of the pharmaceutical before administration, may be fixed to one or more specific people, or, for example, may be one or more people selected as needed from among a number of people on the basis of work shifts and the like. Also, the dosing person may be the administrator, and conversely the administrator may be the dosing person.

Consequently, on the administrator side, the type and dose of the pharmaceutical that will actually be administered to the patient are displayed on the basis of the information about the type and dose of the specific pharmaceutical received at the terminal device (such as a smartphone, a tablet terminal, a PC, or a biometric information measurement device), and the user can refer to the biometric information measurement result to determine suitability. Instructions from the doctor (the prescription determined by the doctor according to the patient's condition) is preferably displayed along with the pharmaceutical.

As a result, the administrator can be prompted to confirm the type and dose of the specific pharmaceutical set on the basis of the biometric information measurement result, and once this determination result by the administrator is obtained, the dosing person can reliably carry out the administration of the specific pharmaceutical as dictated by the biometric information measurement result.

### Effects

With the pharmaceutical injection assessment system according to the present invention, an administrator is prompted to confirm the type and dose of a specific pharmaceutical set on the basis of a biometric information measurement result, and once this determination result by the administrator is obtained, the dosing person can reliably carry out the administration of the specific pharmaceutical as dictated by the biometric information measurement result.

### BRIEF DESCRIPTION OF DRAWINGS

FIG. 1 is a block diagram of the configuration of the pharmaceutical injection assessment system according to one embodiment of the present invention;

FIG. 2 shows the usage state of a blood glucose level measurement device included in the pharmaceutical injection assessment system of FIG. 1;

FIG. 3 is a block diagram of the configuration of the blood glucose level measurement device included in the pharmaceutical injection assessment system of FIG. 1;

FIG. 4 is a block diagram of the configuration of a smartphone on the dosing person side, included in the pharmaceutical injection assessment system of FIG. 1;

FIG. 5 is a block diagram of the configuration of a terminal device on the administrator side, included in the pharmaceutical injection assessment system of FIG. 1;

FIGS. 6A to 6C show data, etc., stored in the memory units provided to the blood glucose level measurement device, the smartphone, and the terminal device included in the pharmaceutical injection assessment system of FIG. 1;

FIG. 7 is a confirmation image captured by the smartphone in FIG. 4, and shows an image of a syringe having graduations showing the amount of insulin that will actually be administered;

FIG. 8 is a confirmation image captured by the smartphone of FIG. 4, and shows an image of a wristband including information such as an ID of the patient to whom insulin will actually be administered;

FIG. 9 is a flowchart showing the processing flow of the pharmaceutical injection determination method on the dosing person side, which is carried out in the pharmaceutical injection assessment system of FIG. 1;

FIG. 10 is a flowchart showing the processing flow of the pharmaceutical injection determination method on the administrator side, which is carried out in the pharmaceutical injection assessment system of FIG. 1;

FIG. 11 is a flowchart showing the processing flow of the pharmaceutical injection determination method on the dosing person side, which is carried out in the pharmaceutical injection assessment system of FIG. 1;

FIGS. 12A to 12D show various messages and the like displayed on the display unit of the blood glucose level measurement device of FIG. 3;

FIGS. 13A and 13B show various messages and the like displayed on the display of the terminal device of FIG. 5;

FIGS. 14A to 14D show various messages and the like displayed on the display unit of the blood glucose level measurement device of FIG. 3;

FIG. 15 is a block diagram of the configuration of the blood glucose level measurement device included in the pharmaceutical injection assessment system according to another embodiment of the present invention; and

FIG. 16 shows the configuration of a pharmaceutical injection pump that includes an insulin pump for administering insulin to a patient on the basis of the determination result in the pharmaceutical injection assessment system according to yet another embodiment of the present invention.

### DESCRIPTION OF EMBODIMENTS

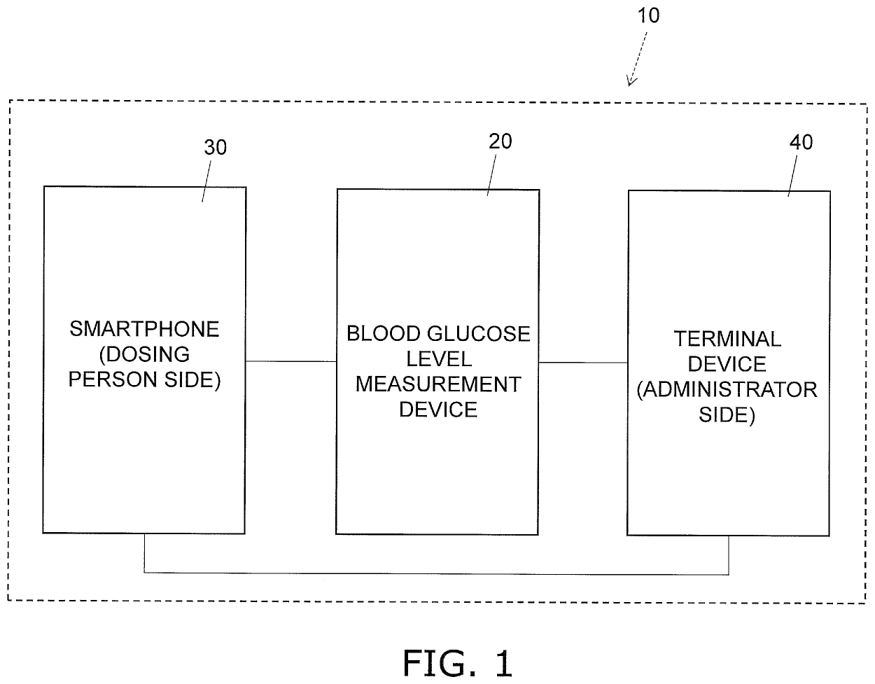

The pharmaceutical injection assessment system 10 according to an embodiment of the present invention will now be described with reference to FIGS. 1 to 14D.

(1) Configuration of Pharmaceutical Injection Assessment System 10

The pharmaceutical injection assessment system 10 according to this embodiment is a system with which the suitability of the dose of insulin that will actually be administered is determined by an administrator other than the dosing person on the basis of the measurement result of the blood glucose level of a patient with diabetes or the like, measured by a nurse or the like using a blood glucose level measurement device 20. As shown in FIG. 1, the pharmaceutical injection assessment system 10 comprises the blood glucose level measurement device 20 on the dosing person side, a smartphone 30 belonging to the dosing person, and a terminal device 40 on the administrator side.

In this embodiment, let us assume that the measurer who measures the blood glucose level (biometric information) of a patient A using the blood glucose level measurement device (biometric information measurement device) 20 included in the pharmaceutical injection assessment system 10 is the same person (nurse B, etc.) as the dosing person who uses a syringe 51 to administer insulin (the specific pharmaceutical), which is appropriate for the measurement result, to the patient A. Let us also assume that the administrator who determines whether or not the dose of insulin that will actually be administered to patient A is appropriate is a different person from the nurse B (another nurse C1, a head nurse C2, a doctor D, etc.).

(2-1) Blood Glucose Level Measurement Device 20

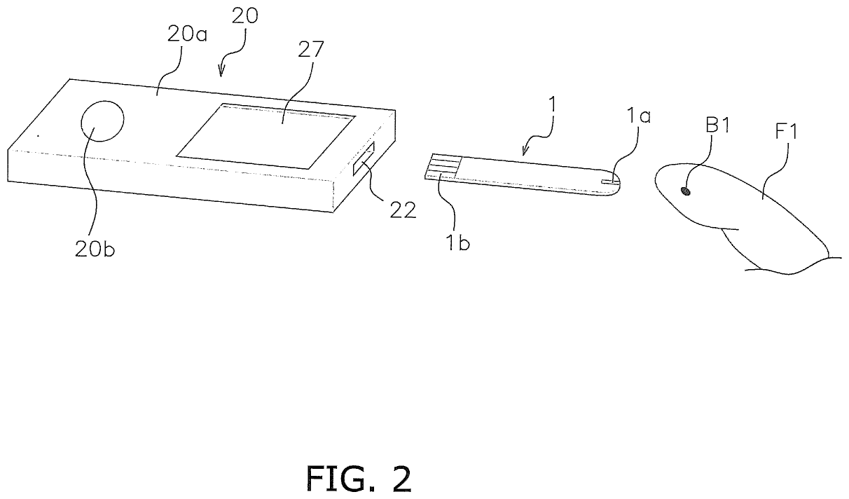

The blood glucose level measurement device 20 in this embodiment is, for example, a biometric information measurement device a plurality of which are used in the same medical department, on the same floor, or the like, and as shown in FIG. 2, for example, measures the blood glucose level (biometric information) in a state in which a sensor 1, on which a drop of blood B1 discharged from a puncture wound made in the patient's finger F1 has been applied, has been inserted.

Here, as shown in FIG. 2, the sensor 1 mounted in a sensor insertion slot (sensor mounting portion) 22 of the blood glucose level measurement device 20 has a deposition port 1a and an electrode portion 1b.

The deposition port 1a is provided at the first end portion of the sensor 1. When the blood B1 flowing out of the patient's finger F1 is deposited in the deposition port 1a, the blood B1 moves under capillary action in a specific direction along the supply path in the sensor 1, and reacts with a reagent layer (not shown) provided inside the sensor 1.

The electrode portion 1b is provided at the second end portion of the blood glucose level measurement device 20 on the side where this electrode portion 1b is inserted into the sensor insertion slot 22. The above-mentioned reagent layer includes a redox enzyme and an electron acceptor, and is disposed in the sensor 1 so as to cover a part of the electrode portion 1b. When the electrode portion 1b is inserted into the sensor insertion slot 22, the sensor 1 and the blood glucose level measurement device 20 are electrically connected so as to measure the current obtained by the electrochemical oxidation of the electron acceptor included in the reagent layer as a result of an electrochemical reaction between the blood B1 and the reagent layer.

Consequently, the concentration of glucose in the blood (blood glucose level) can be measured by using the blood glucose level measurement device 20.

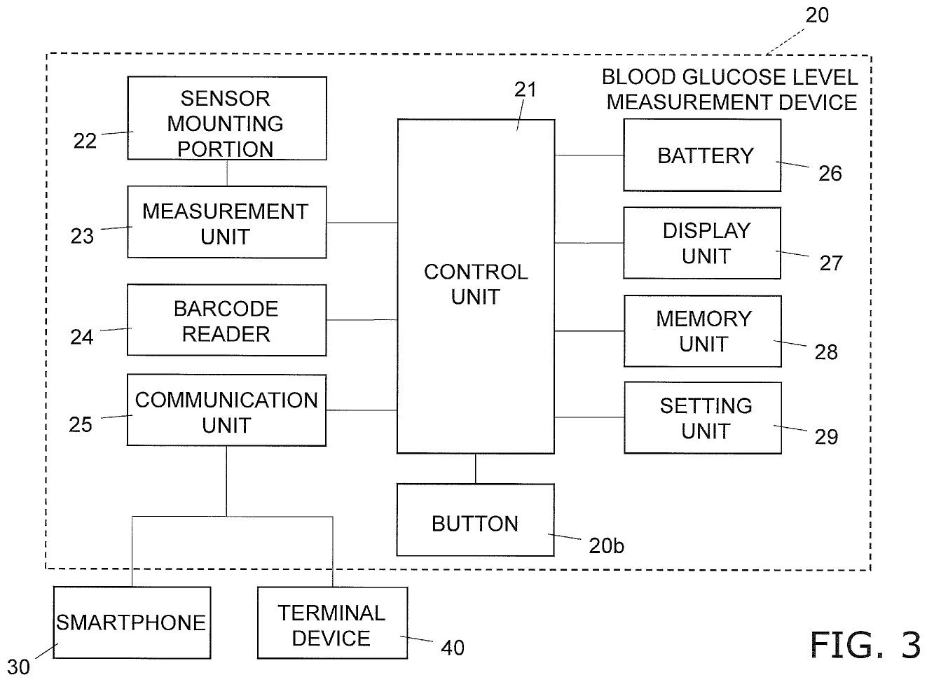

As shown in FIGS. 2 and 3, the blood glucose level measurement device 20 comprises a main body unit 20a, a button 20b, a control unit 21, the sensor insertion slot (sensor mounting unit) 22, a measurement unit 23, a barcode reader (information reading unit) 24, a communication unit (pharmaceutical dose acquisition unit, first communication unit) 25, a battery 26, a display unit 27, a memory unit 28, and a setting unit 29.

The main body unit 20a is the case portion of the blood glucose level measurement device 20, and houses in its interior the control unit 21, the measurement unit 23, the barcode reader 24, the communication unit 25, the battery 26, the display unit 27, the memory unit 28, and the setting unit 29 shown in FIG. 3.

The button 20b is an operation unit that is operated when starting the measurement of the blood glucose level, etc., and is provided on the surface of the main body unit 20a where the display unit 27 is provided.

As shown in FIG. 3, the control unit 21 is connected to the measurement unit 23, the barcode reader 24, the communication unit 25, the button 20b, the battery 26, the display unit 27, the memory unit 28, and the setting unit 29.

The sensor insertion slot 22 is an opening formed in the end surface of the main body unit 20a, and the sensor 1 is mounted therein.

As shown in FIG. 3, the measurement unit 23 is connected to a connector (not shown) of the sensor insertion slot 22. The measurement unit 23 measures the current obtained by the electrochemical oxidation of the electron acceptor included in the reagent layer as a result of the reaction between the blood B1 and the reagent layer in the electrode portion 1b of the sensor 1 connected to the sensor insertion slot 22.

The barcode reader (information reading unit) 24 is provided, for example, on the rear surface side of the main body unit 20a, and during measurement of the blood glucose level (discussed below), the patient ID, user ID, pharmaceutical ID, sensor bottle ID, and so forth are read and transmitted to the control unit 21.

As shown in FIG. 3, the communication unit (pharmaceutical dose acquisition unit, first communication unit) 25 is connected to the smartphone 30 belonging to the dosing person and to the terminal device 40 on the administrator side. The communication unit 25 then transmits information such as the blood glucose level measured by the blood glucose level measurement device 20, the patient who measured the blood glucose level, the nurse, the pharmaceutical (insulin), and so forth to the smartphone 30 and the terminal device 40.

The battery 26 is, for example, a rechargeable battery, and is provided as the power supply for the blood glucose level measurement device 20. The battery 26 drives the blood glucose level measurement device 20 by supplying a substantially constant current to the control unit 21 and the like.

The display unit 27 is a liquid crystal display panel provided on the front of the main body unit 20a (the sensor insertion slot 22 side), and displays messages indicating the result of measuring the blood glucose level, the result of suitability determination in the actual administration of insulin to a patient, or the like.

The memory unit 28 is a storage means for storing various types of data, and is connected to the control unit 21.

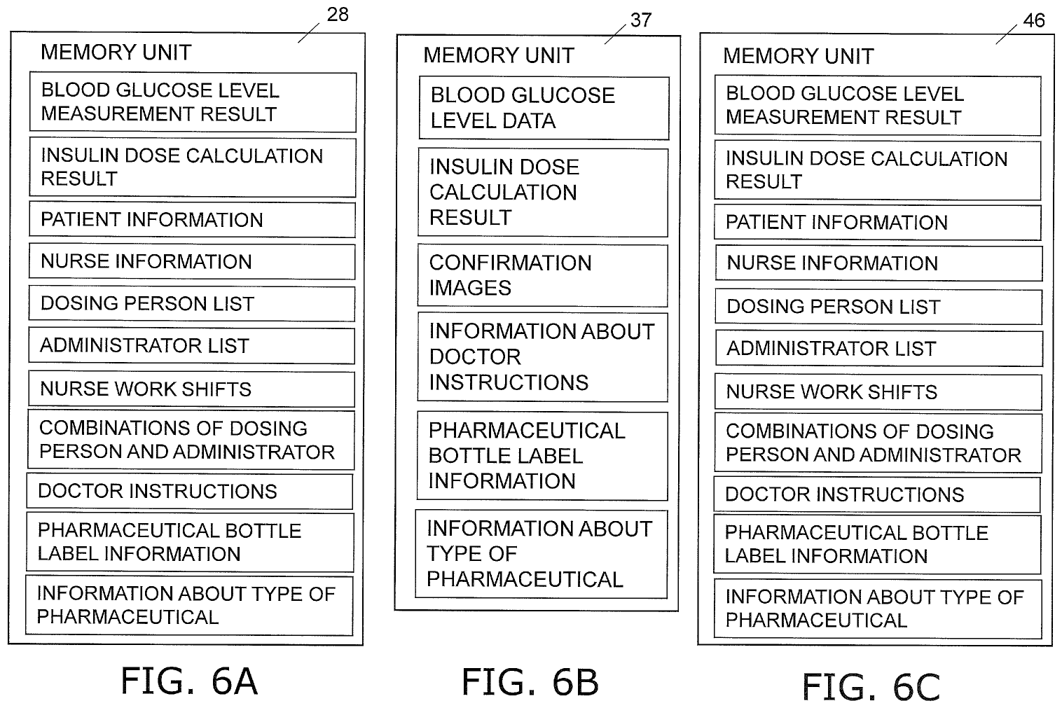

As shown in FIG. 6A, for example, the memory unit 28 stores blood glucose level measurement results, insulin doses calculated on the basis of a measurement result, patient information collated with patient IDs read by the barcode reader 24, information about nurses serving as the dosing person, various messages that are displayed when measuring blood glucose level, confirmation images (discussed below), a dosing person list, an administrator list, nurse work shifts, data about combinations of dosing person and administrator, doctor instructions, pharmaceutical bottle label information, information about the type of pharmaceutical, and various other types of information.

For example, when a nurse working in a hospital scans his or her nurse ID with the barcode reader 24, the setting unit 29 refers to the dosing person list, administrator list, and nurse work shifts stored in a memory unit 28, and automatically matches the nurse whose nurse ID was read with the administrator who will be the dosing person.

That is, the setting unit 29 sets the combination of dosing person and administrator so as to make an appropriate combination from among all the nurses, etc., who are working that day.

Consequently, the nurse on the dosing person side can recognize the person (doctor, nurse, etc.) who has been automatically set on the administrator side by taking into account the work shifts that day, by scanning his or her own nurse ID with the barcode reader 24 of the blood glucose level measurement device 20 at the start of work, etc.

Information related to the combination of dosing person and administrator set by the setting unit 29 can be transmitted from the communication unit 25 of the blood glucose level measurement device 20 and shared with other blood glucose level measurement devices used in the same medical department or on the same floor, or with a smartphone 30 belonging to the dosing person and the terminal device 40 on the administrator side.

Here, the combination of dosing person and administrator set by the setting unit 29 is not limited to a combination of one person with one person, and may instead be a combination of one person with multiple people, multiple people with one person, or multiple people with multiple people.

(2-2) Smartphone 30

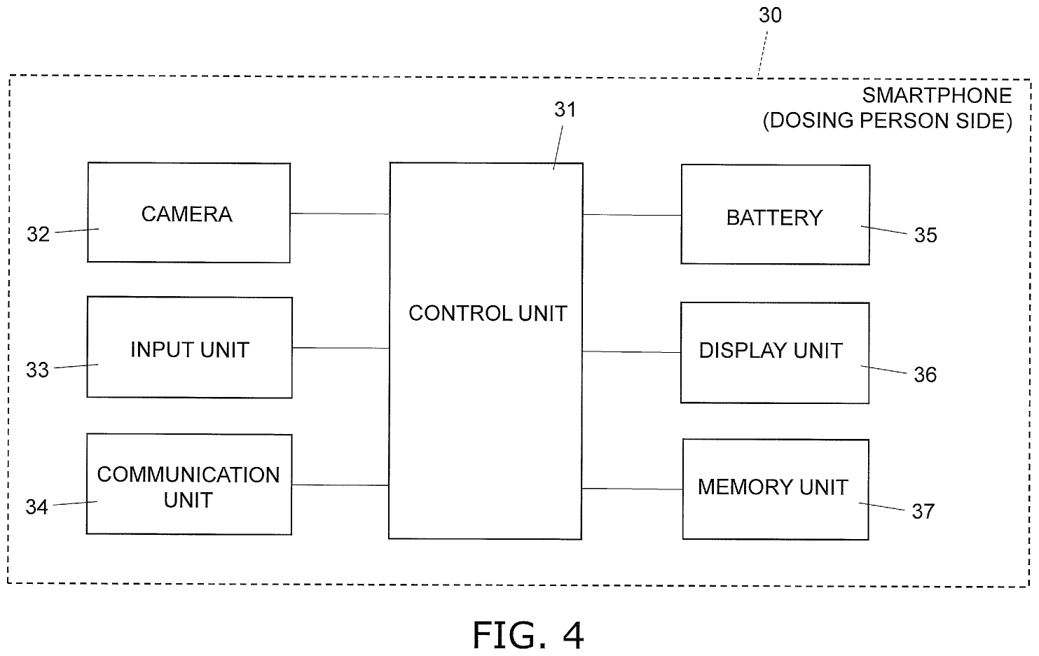

The smartphone 30 is a communication terminal with a camera function belonging to the nurse serving as the dosing person, and is used to capture a confirmation image for the purpose of determining the suitability of the insulin dose (discussed below). More specifically, as shown in FIG. 4, the smartphone 30 comprises a control unit 31, a camera (imaging unit) 32, an input unit 33, a communication unit 34, a battery 35, a display unit 36, and a memory unit 37.

As shown in FIG. 4, the control unit 31 is connected to the camera 32, the input unit 33, the communication unit 34, the battery 35, the display unit 36, and the memory unit 37. The control unit 31 receives electric power from the battery 35 and controls various components in order to carry out the various functions of the smartphone 30.

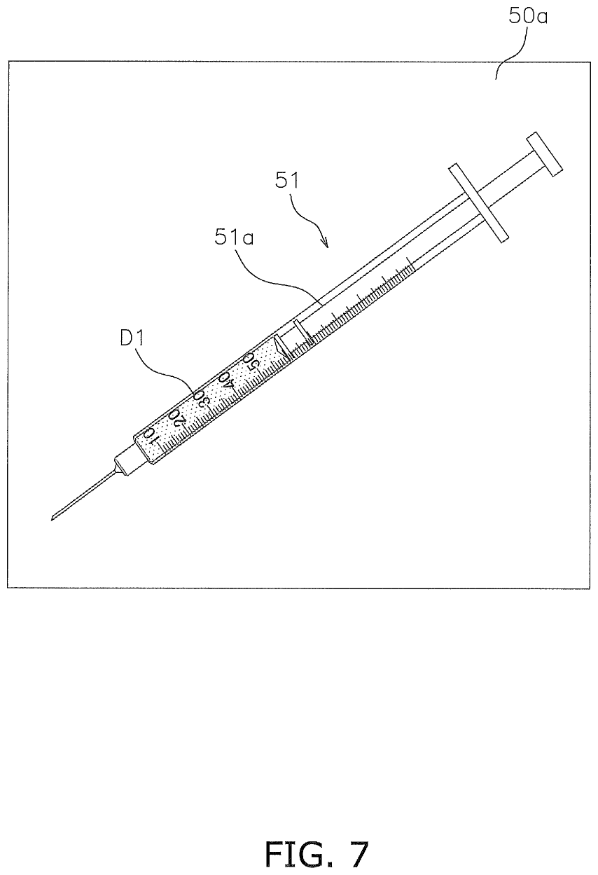

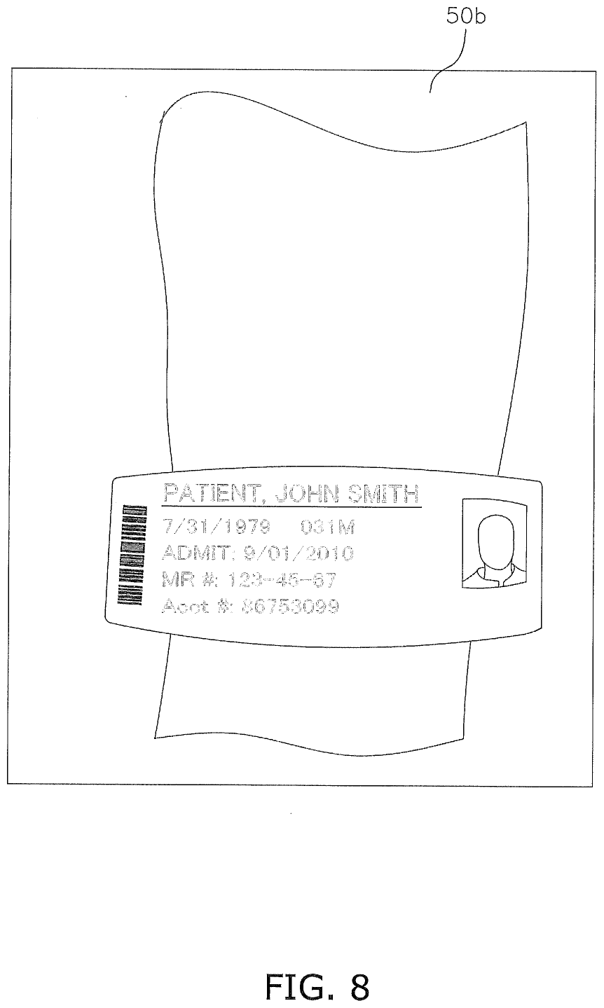

The camera (imaging unit) 32 is an imaging device capable of capturing a still pictures or moving pictures, and in this embodiment captures an image including a graduated portion 51a indicating the actual insulin dose that was put in the syringe 51 on the basis of the measurement result for blood glucose level (see FIG. 7), an image of a wristband including patient information (see FIG. 8), or other such confirmation images 50a and 50b.

The confirmation images 50a and 50b showing the state prior to administration of insulin or another such pharmaceutical are captured at a timing such as immediately before the pharmaceutical injection, immediately before the pharmaceutical injection button is pressed, or immediately before the pharmaceutical injection button is operated.

The input unit 33 is, for example, a touch panel of the display unit 36 of the smartphone 30, and various operation and setting information, etc., is inputted.

The communication unit 34 receives the blood glucose level data measured by the blood glucose level measurement device 20 from the communication unit 25. The communication unit 34 can connect to the Internet, and for example, and transmits data about the appropriate insulin dose for the measured blood glucose level calculated using the calculation software on the network in the smartphone 30, together with the confirmation images 50a and 50b captured by the camera 32, to the communication unit 44 of the terminal device 40 on the administrator side.

The battery 35 is built into the smartphone 30, and a secondary battery that can be repeatedly charged is used, for example. The battery 35 supplies electric power to the various components of the control unit 31 and the like installed in the smartphone 30.

The display unit 36 is, for example, a touch panel-type liquid crystal display panel, and displays various types of information, such as blood glucose level data received from the blood glucose level measurement device 20, and the confirmation images 50a and 50b captured by the camera 32.

The memory unit 37 stores various types of image data such as the confirmation images 50a and 50b captured by the camera 32, various types of data such as the blood glucose level received via the communication unit 34, and other such information.

As shown in FIG. 6B, for example, the memory unit 37 stores the blood glucose level data received via the communication unit 34, the insulin dose calculated on the basis of a measurement result, various types of image data including the confirmed confirmation images 50a and 50b captured by the camera 32, information about doctor instructions, pharmaceutical bottle label information, and pharmaceutical type information.

(2-3) Terminal Device 40

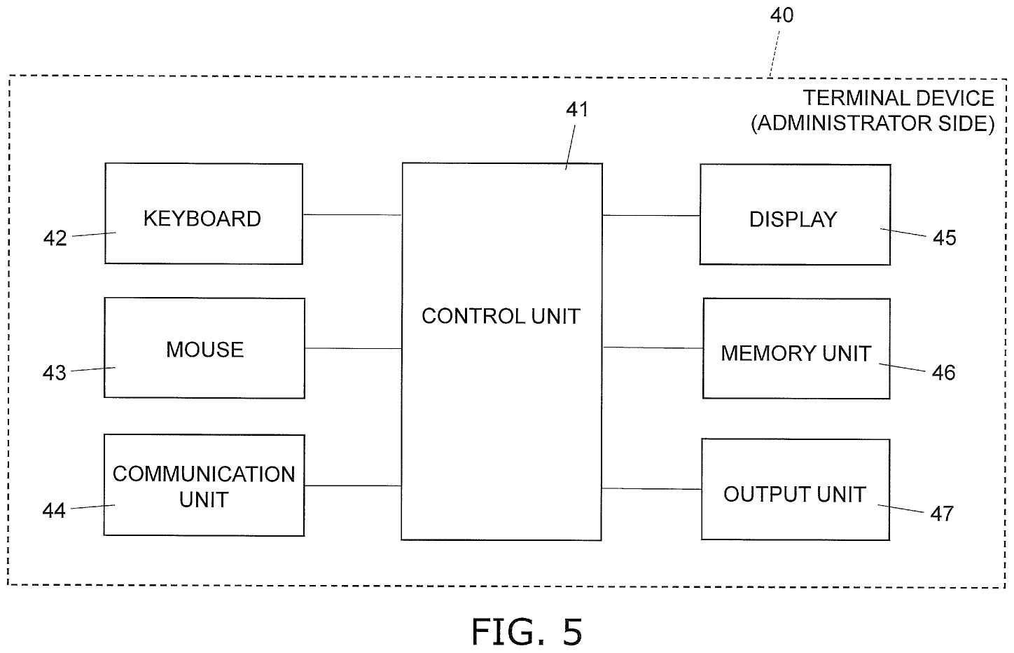

The terminal device 40 is a PC, a tablet terminal, a biometric information measurement device, or the like that is operated by an administrator who determines the suitability of the above-mentioned insulin administration, and as shown in FIG. 5, comprises a control unit 41, a keyboard (input unit) 42, a mouse (input unit) 43, a communication unit (reception unit, second communication unit) 44, a display (display unit) 45, a memory unit 46, and an output unit 47.

As shown in FIG. 5, the control unit 41 is connected to the keyboard 42, the mouse 43, the communication unit 44, the display 45, the memory unit 46, and the output unit 47. The control unit 41 controls that various components in order to carry out the various functions as a PC.

The keyboard (input unit) 42 functions as an input unit for inputting text information and so forth.

The mouse (input unit) 43 functions as a selection means for selecting the input position of text information inputted with the keyboard 42 and various other types of information on the selection screen, and also functions as an input unit for inputting the result of a suitability determination regarding the administration of insulin (discussed below).

The communication unit (reception unit, second communication unit) 44 receives data about the appropriate insulin dose for the blood glucose level measured by the blood glucose level measurement device 20 and the confirmation images 50a and 50b used for determining the suitability of insulin administration, from the communication unit 34 of the smartphone 30. Furthermore, the communication unit 44 receives data about the blood glucose level measured by the measurement unit 23, information about the patient for whom the blood glucose level was measured, nurse information, information about the pharmaceutical to be administered (insulin), the combination of dosing person and administrator set in the setting unit 29, and other such information, from the communication unit 25 of the blood glucose level measurement device 20.

The display (display unit) 45 is, for example, a liquid crystal display for a PC, and displays various types of information received via the communication unit 44, as well as a determination screen and so forth for when determining the suitability of insulin administration (discussed below) on the administrator side.

The memory unit 46 is a storage means for storing various types of data, and is connected to the control unit 21.

As shown in FIG. 6C, for example, the memory unit 46 stores the blood glucose level measurement result, the dose of insulin calculated on the basis of the measurement result, patient information collated with the patient ID read by the barcode reader 24, information about the nurse serving as the dosing person, various messages that are displayed when measuring blood glucose level, confirmation images (discussed below), a dosing person list, an administrator list, nurse work shifts, data about combinations of dosing person and administrator, doctor instructions, pharmaceutical bottle label information, information about the type of pharmaceutical, and various other kinds of information.

That is, for example, the memory unit 46 stores information that is similar to the various types of information stored in the memory unit 28 of the blood glucose level measurement device 20, and information is shared between the plurality of blood glucose level measurement devices 20 used in the same medical department, etc., and the terminal devices 40 connected to these blood glucose level measurement devices 20, via wireless communication.

Consequently, for example, if data related to the combination of dosing person and administrator set in the setting unit 29 of the blood glucose level measurement device 20 is shared with the terminal device 40 on the administrator side or with a plurality of other blood glucose level measurement devices 20, confirmation can be performed from any device.

When the terminal device 40 on the administrator side receives a confirmation image or the like from the smartphone 30, the output unit 47 promptly outputs voice information or light that prompts the user to make a determination regarding insulin administration.

Consequently, a determination as to the suitability of the administration of insulin and to the insulin dose can be made promptly on the administrator side, without making the dosing person or patient who are on standby immediately prior to the actual administration wait any longer than necessary, and the determination result can be sent back to the blood glucose level measurement device 20 or the smartphone 30 on the dosing side.

If the terminal device 40 on the administrator side is a tablet terminal or the like, the output unit 47 may be configured to vibrate, rather than outputting sound or light.

Consequently, the administrator can recognize that the suitability of insulin administration should be determined, and can promptly send the result of the suitability determination back to the blood glucose level measurement device 20 or the smartphone 30 on the dosing person side.

### Determination Suitability Processing Related to Actual Administration of Insulin

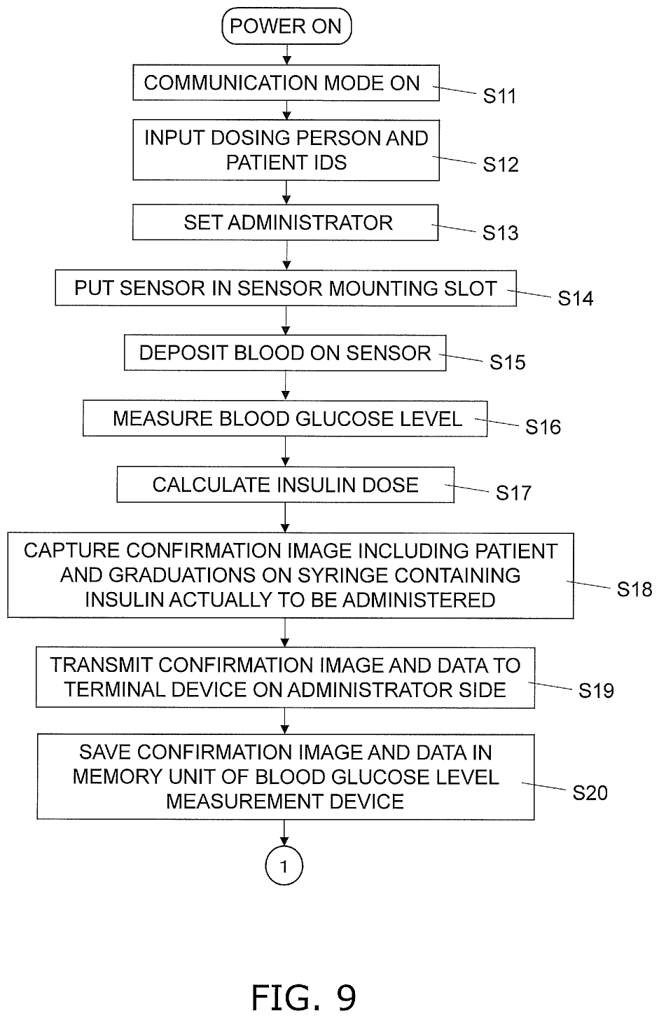

The flow of the processing to determine the suitability of insulin administration carried out on the administrator side and related to insulin administration that will be carried out by the pharmaceutical injection assessment system 10 in this embodiment will now be described using the flowcharts of FIGS. 9 to 11.

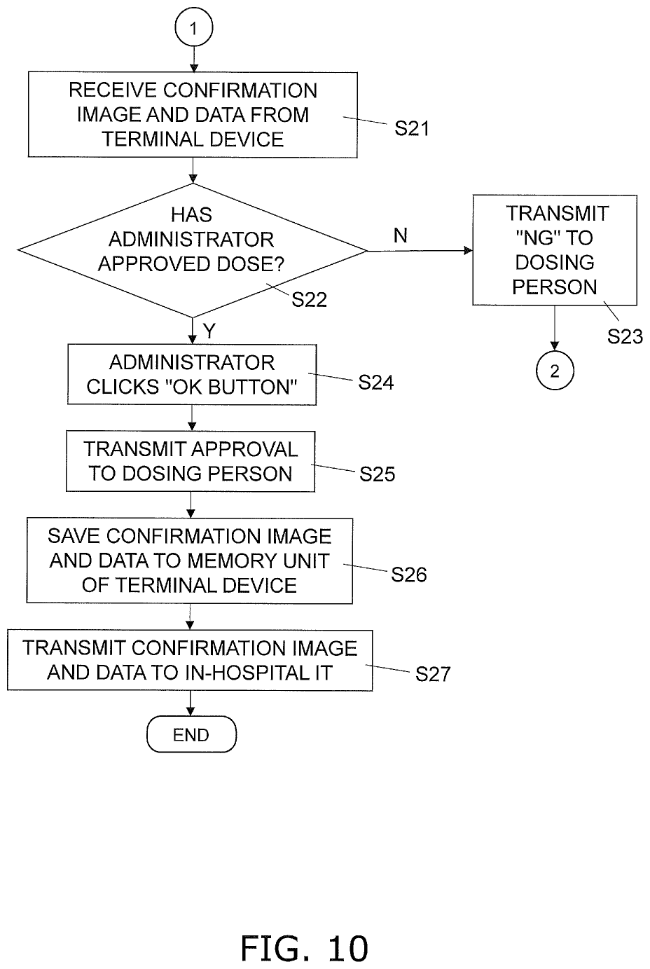

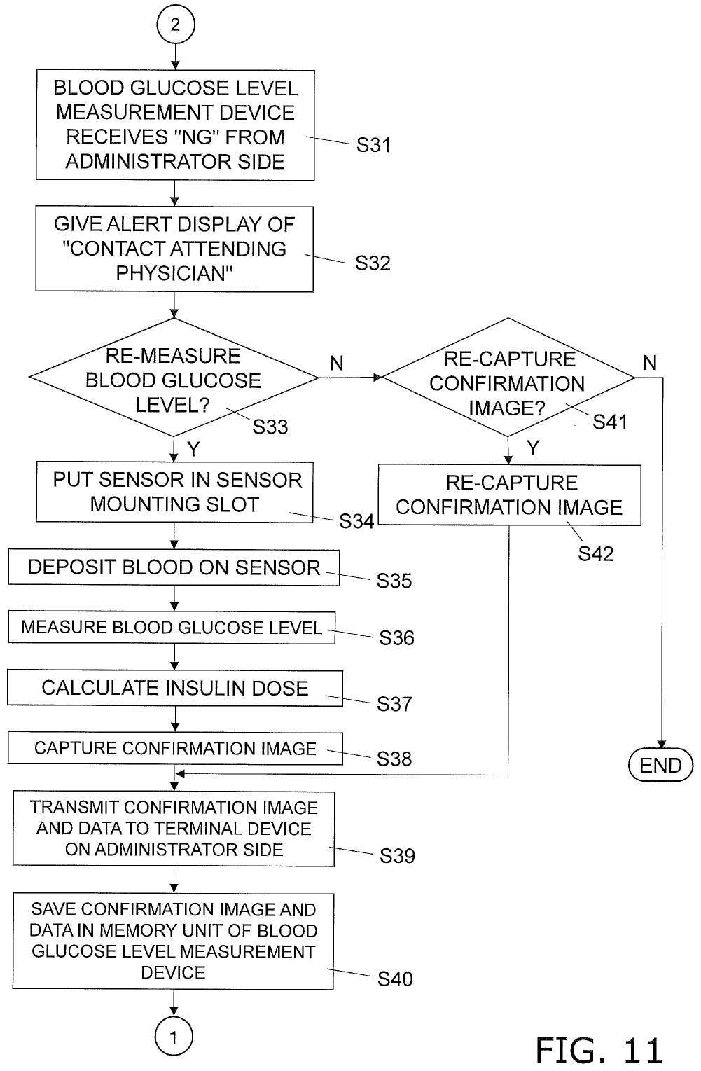

FIGS. 9 and 11 show the processing on the blood glucose level measurement device 20 side, and FIG. 10 shows the processing at the terminal device 40 on the administrator side.

That is, when the blood glucose level measurement device 20 is turned on, the battery 26 starts supplying power, and in step S11 of the flowchart of FIG. 9, the control unit 21 turns on the communication mode so as to allow communication via the communication unit 25.

Next, in step S12, a nurse or other such dosing person uses the barcode reader 24 to input his or her nurse ID, the patient ID, the bottle ID of insulin to be administered to the patient (information related to the specific pharmaceutical type), and so forth.

Information related to the type of pharmaceutical may be acquired, for example, by using an image obtained by imaging the label portion of the pharmaceutical bottle of the specific pharmaceutical (such as insulin) that has been drawn in by a syringe.

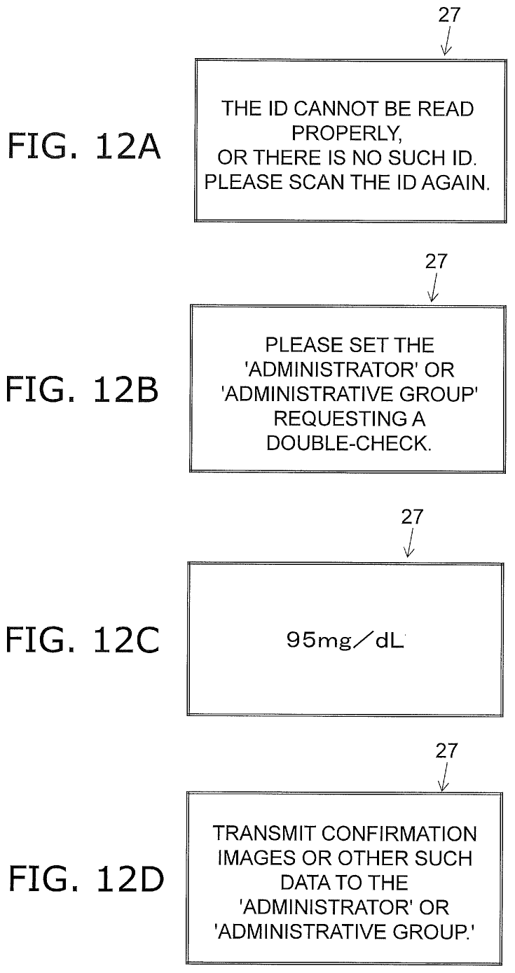

If ID input with the barcode reader 24 is not carried out properly, as shown in FIG. 12A, the control unit 21 will cause the display screen of the display unit 27 to display a message saying “The ID cannot be read properly, or there is no such ID. Please scan the ID again” to prompt re-input.

Also, the memory unit 28 may store the range (upper limit value and lower limit value) of the dose recommended by the doctor regarding the dose of insulin corresponding to the patient ID read by the barcode reader 24. In this case, the communication unit 25 transmits the doctor's instruction matching the patient ID (EMR; electronic medical record) to the terminal device 40 on the administrator side, which allows the administrator to determine the suitability of the insulin dose in light of the instructions from the doctor.

Next, in step S13, the setting unit 29 sets the nurse, etc., whose ID has been read by the barcode reader 24 as the dosing person, and also automatically selects the administrator who will determine the appropriateness of insulin administration from the list saved in the memory unit 28.

If automatic setting by the setting unit 29 is turned off, the control unit 21 may cause the display screen of the display unit 27 to display a message that says “Please set the ‘administrator’ or ‘administrative group’ requesting a double-check,” as shown in FIG. 12B.

Thus, when the dosing person manually sets a combination with an administrator, the combination of dosing person and administrator is may be set, for example, by selecting from a pull-down display of the names, IDs, and so forth of a plurality of nurses, doctors, etc., that have been pre-registered on the setting screen of the blood glucose level measurement device 20.

The registration of the dosing person and the administrator may be inputted using the terminal device 40 on the administrator side in which an in-hospital data management system (DMS) has been installed, for example. Consequently, the POC (point of care) coordinator (POCC) can share registrants by connecting a plurality of blood glucose level measurement devices 20 used in the same medical department or on the same floor to the terminal device 40 in a clinical laboratory.

Alternatively, when a dosing person and an administrator are registered in the blood glucose level measurement device 20, the registered data may be transmitted to the DMS and managed by the POCC in the clinical laboratory.

Furthermore, instead of using a DMS, if software that matches up a dosing person with an administrator is installed on the PC, etc., for each medical department in the hospital, the matching of the dosing person and the administrator can be managed by using this PC.

Next, in step S14, the sensor 1 is put in the sensor insertion slot 22 of the blood glucose level measurement device 20 by the dosing person in order to measure the blood glucose level.

Next, in step S15, blood that comes out of a puncture wound made in a patient's fingertip or the like using a puncture device (not shown), for example, is deposited in the deposition port 1a of the sensor 1.

Next, in step S16, the measurement unit 23 uses the blood deposited on the sensor 1 to measure the blood glucose level.

At this point, as shown in FIG. 12C, the measurement result data of, such as “95 mg/dL,” is displayed on the display unit 27 of the blood glucose level measurement device 20.

Next, in step S17, the communication unit 25 of the blood glucose level measurement device 20 transmits the measurement result (blood glucose level data) obtained by the measurement unit 23 to the smartphone 30, and the control unit 31 of the smartphone 30 calculates the optimal insulin dose for the measurement result by using calculation software that is on the Internet. Then, the calculated insulin dose is transmitted from the communication unit 34 of the smartphone 30 to the communication unit 25 of the blood glucose level measurement device 20.

Next, in step S18, the camera 32 of the smartphone 30 belonging to the dosing person is used to capture a confirmation image 50a (see FIG. 7) including a portion of the graduations on the syringe 51 indicating the dose of insulin D1 that will actually be administered to the patient, which was put in the syringe 51 after the blood glucose level measurement device 20 confirmed the calculated insulin dose received from the smartphone 30. Furthermore, in step S18, the camera 32 of the smartphone 30 belonging to the dosing person is used to capture an image of the patient's name tag, which includes information such as the patient's name (John Smith), admitted date (ADMIT), attending physician ID (MR #), patient ID (account No.), medical department, ward, etc., as the confirmation image 50b (see FIG. 8) including information about the patient to whom the insulin D1 contained in the syringe 51 will be administered.

If the blood glucose level measured in step S16 is within the normal range, it is assumed that the insulin dose calculated in step S17 is “0.” In this case, an empty syringe, which means that the insulin dose is zero, may be imaged as a confirmation image. Consequently, the administrator can determine from the measurement result of the blood glucose level whether or not an insulin dose of zero is appropriate.

Next, in step S19, the confirmation images 50a and 50b taken by the camera 32 of the smartphone 30, the blood glucose level data measured by the blood glucose level measurement device 20, and the insulin dose data calculated by the smartphone 30 and acquired by the blood glucose level measurement device 20 is transmitted to the terminal device 40 on the administrator side.

At this point, as shown in FIG. 12D, the display unit 27 of the blood glucose level measurement device 20 displays a message that says “Transmit confirmation images or other such data to the ‘administrator’ or ‘administrative group.’”

Next, in step S20, in the blood glucose level measurement device 20, the control unit 21 causes the memory unit 28 to store the confirmation images 50a and 50b, the blood glucose level measurement result, the insulin dose calculation result, and other such data.

Next, the processing of steps S21 to S27 in the flowchart of FIG. 10 is carried out by the terminal device 40 on the administrator side.

That is, in step S21, the communication unit 44 of the terminal device 40 receives the confirmation images 50a and 50b, the blood glucose level measurement result, the insulin dose calculation result, and other such data from the dosing person side (blood glucose level measurement device 20 and smartphone 30).

Next, in step S22, the control unit 41 causes the display 45 of the terminal device 40 to display the confirmation images 50a, 50b, etc., and determines whether or not the administrator has approved the dose.

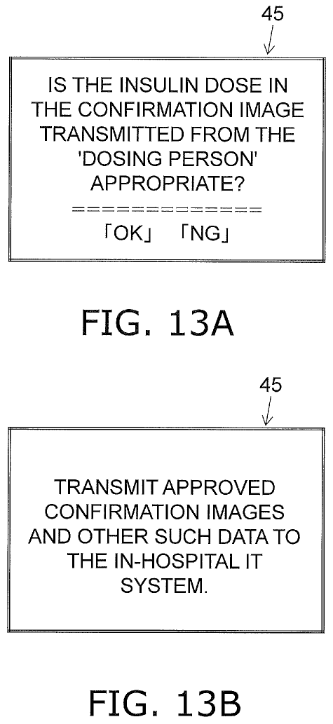

At this point, as shown in FIG. 13A, the display 45 displays a message that says “Is the insulin dose in the confirmation image transmitted from the ‘dosing person’ appropriate?,” and the administrator can select “OK” or “NG” displayed on the display 45.

Here, if the insulin dose is approved by the administrator, the processing proceeds to step S24, and if the dose is not approved, the processing proceeds to step S23.

Next, in step S23, since the administrator did not approve in step S22, a reply of “NG” (cannot be approved) is transmitted to the dosing side (the blood glucose level measurement device 20 and the smartphone 30).

Next, in step S24, since the administrator approved in step S22, the “OK button” is clicked.

Next, in step S25, a reply of “approved” is transmitted to the dosing person side (the blood glucose level measurement device 20 and the smartphone 30).

Next, in step S26, the control unit 41 causes the memory unit 46 of the terminal device 40 to store the confirmation images 50a and 50b, the blood glucose level measurement result, the insulin dose calculation result, and other such data.

Next, in step S27, the control unit 41 transmits the confirmation images 50a and 50b, the blood glucose level measurement result, the insulin dose calculation result, and other such data via the communication unit 44 to an in-hospital IT (information technology) system.

At this point, as shown in FIG. 13B, the message that says “Transmit approved confirmation images and other such data to the in-hospital IT system” is displayed on the display 45 of the terminal device 40.

Consequently, a confirmation image taken at the time of insulin administration, a blood glucose level measurement result, an insulin calculation result, and other such data are stored in the in-hospital database, and can be used as evidence for confirmation at a later date.

Next, the processing in steps S31 to S42 in the flowchart of FIG. 11 is performed again by the blood glucose level measurement device 20 on the dosing person side.

That is, in step S31, the communication unit 25 of the blood glucose level measurement device 20 receives a message of “NG” transmitted from the terminal device 40 on the administrator side in S23.

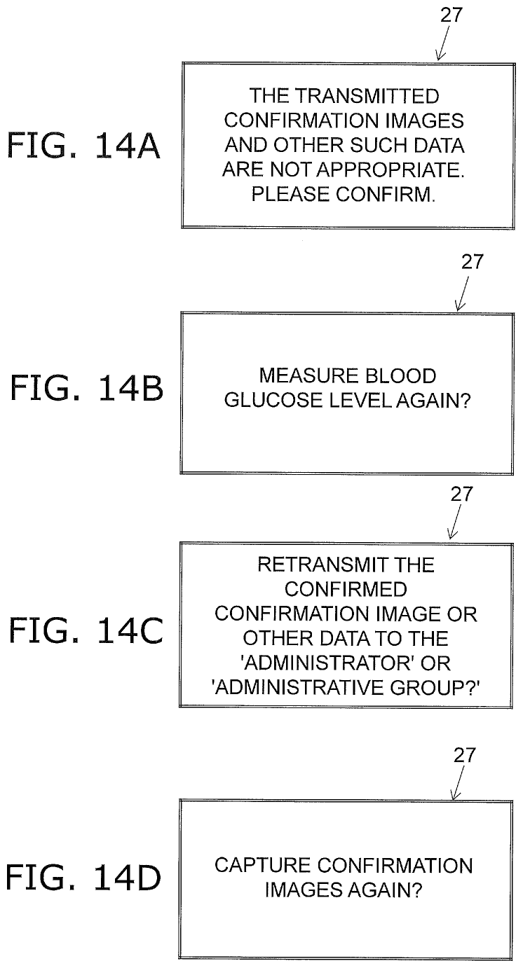

At this point, the display unit 27 of the blood glucose level measurement device 20 displays a message that says “The transmitted confirmation images and other such data are not appropriate. Please confirm,” as shown in FIG. 14A, for example.

Next, in step S32, an alert display of “Contact the attending physician” is displayed on the display unit 27.

Next, in step S33, it is determined whether or not to measure the blood glucose level again.

At this point, as shown in FIG. 14B, a message that says “Measure blood glucose level again?” is displayed on the display unit 27 of the blood glucose level measurement device 20.

Here, if the dosing person has chosen to re-measure the blood glucose level, the processing proceeds to step S34. On the other hand, if the dosing person does not choose to re-measure the blood glucose level, the processing proceeds to step S41.

Next, in step S34, since the re-measurement of the blood glucose level was selected in the step S33, the dosing person puts a new sensor 1 in the sensor insertion slot 22 of the blood glucose level measurement device 20 in order to measure the blood glucose level again.

Next, in step S35, the blood that comes out from a puncture wound made in the fingertip or the like of the patient using a puncture device (not shown) is again deposited in the deposition port 1a of the sensor 1.

Next, in step S36, the measurement unit 23 uses the blood deposited on the sensor 1 to measure the blood glucose level.

Next, in step S37, the communication unit 25 of the blood glucose level measurement device 20 transmits the measurement result (blood glucose level data) measured by the measurement unit 23 to the smartphone 30, and the control unit 31 of the smartphone 30 uses calculation software on the Internet to calculate the optimal insulin dose with respect to the measurement result. The calculated insulin dose is then transmitted from the communication unit 34 of the smartphone 30 to the communication unit 25 of the blood glucose level measurement device 20.

Next, in step S38, the camera 32 of the smartphone 30 belonging to the dosing person is again used to capture a confirmation image 50a (see FIG. 7) including a portion of the graduated portions 51a on the syringe 51 indicating the dose of insulin D1 that will actually be administered to the patient and that was put in the syringe 51 after confirmation of the calculated insulin dose received by the blood glucose level measurement device 20 from the smartphone 30. Furthermore, in step S38, as in step S18, the camera 32 of the smartphone 30 belonging to the dosing person is used to capture an image of the patient's name tag, including the patient's name and ID, the medical department, the ward, and other such information, as the confirmation image 50b (FIG. 8) that includes information about the patient to whom insulin D1 contained in the syringe 51 will be administered.

Next, in step S39, the confirmation images 50a and 50b taken with the camera 32 of the smartphone 30, the blood glucose level data measured with the blood glucose level measurement device 20, and insulin dose data calculated by the smartphone 30 and acquired by the blood glucose level measurement device 20 are again transmitted to the terminal device 40 on the administrator side.

At this point, as shown in FIG. 14C, the display unit 27 of the blood glucose level measurement device 20 displays a message that says “Retransmit the confirmed confirmation image or other data to the ‘administrator’ or ‘administrative group?,’” and if the dosing person inputs “Yes,” the data is retransmitted.

Next, in step S40, in the blood glucose level measurement device 20, the control unit 21 stores the confirmation images 50a and 50b, the blood glucose level measurement result, the insulin dose calculation result, and other such data in the memory unit 28.

The measurement data, confirmation images, and so forth from the first time may be overwritten by the re-measured data, etc., or the first-time data, etc., may be saved as it is in the memory unit 28.

Next, in step S41, since re-measurement of the blood glucose level was not chosen in step S33, it is determined whether or not to capture the confirmation images 50a and 50b again.

Here, if the dosing person selects to capture the images again, the processing proceeds to step S42, but if this is not selected, it is determined that insulin cannot be administered properly, and the processing is ended.

At this point, as shown in FIG. 14D, a message that says “Capture confirmation images again?” is displayed on the display unit 27 of the blood glucose level measurement device 20.

Next, in step S42, the camera 32 of the smartphone 30 is again used to capture a confirmation image 50a (see FIG. 7) including a portion of the graduated portions 51a of the insulin D1 already contained in the syringe 51, and/or a confirmation image (see FIG. 8) including the patient ID and so forth, and the processing proceeds to step S39.

Next, in step S39, the recaptured confirmation images 50a and 50b, the blood glucose level measurement result, and the insulin calculation result, and other such data are again transmitted to the terminal device 40 on the administrator side.

Next, in step S40, the control unit 21 stores the recapture confirmation images 50a and 50b, the blood glucose level measurement result, the insulin calculation result, and other such data in the memory unit 28, and again the processing moves to the terminal device 40 on the administrator side shown in FIG. 10.

As described above, the pharmaceutical injection assessment system 10 of this embodiment comprises the blood glucose level measurement device 20, the smartphone 30, and the terminal device 40. The blood glucose level measurement device 20 measures the blood glucose level and acquires an insulin dose on the basis of the measured blood glucose level. The smartphone 30 captures a confirmation image showing the type and dose of insulin. The terminal device 40 has a communication unit 44 that receives confirmation images, and a display 45 that prompts the administrator to confirm the type and dose of insulin.

Consequently, on the administrator side, whether or not the insulin dose that will actually be administered to the patient is appropriate can be determined by referring to the blood glucose level measurement result, on the basis of the information related to the insulin dose received by the terminal device 40.

As a result, the administrator can be prompted to determine the suitability of the insulin dose set on the basis of the blood glucose level measurement result, and the dosing person can obtain the determination result made by the administrator and thereby reliably administer insulin as dictated by the blood glucose level measurement result.

### OTHER EMBODIMENTS

An embodiment of the present invention was described above, but the present invention is not limited to or by the above embodiment, and various modifications are possible without departing from the gist of the invention.

(A)

In the above embodiment, an example was given in which the present invention was realized as the pharmaceutical injection assessment system 10, but the present invention is not limited to this.

For example, the present invention may be realized as a pharmaceutical injection assessment program in which a computer executes a pharmaceutical injection determination method carried out according to the flowcharts shown in FIGS. 9 to 11.

In this case, since the pharmaceutical injection assessment program is stored in the memory units provided to the biometric information measurement device, the terminal device, etc., the CPU reads the pharmaceutical injection assessment program and the hardware executes the steps of the pharmaceutical injection determination method.

Alternatively, the present invention may be realized as a recording medium in which this pharmaceutical injection assessment program is stored.

(B)

In the above embodiment, an example was given in which the communication unit 25 of the blood glucose level measurement device 20 functioned as a pharmaceutical dose acquisition unit, wherein the communication unit 25 received the appropriate insulin dose calculated by the smartphone 30 belonging to the dosing person on the basis of the blood glucose level measured by the blood glucose level measurement device 20. However, the present invention is not limited to this.

For example, if software for calculating the appropriate insulin dose on the basis of the blood glucose level is installed in the blood glucose level measurement device, then the control unit may calculate the appropriate insulin dose in the blood glucose level measurement device. That is, the configuration may be such that a pharmaceutical dose acquisition unit that calculates the appropriate insulin dose from the measured blood glucose level may be provided within the blood glucose level measurement device.

(C)

In the above embodiment, an example was given in which the camera 32 installed in the smartphone 30 belonging to the dosing person was used to capture a confirmation image showing whether or not the actual dose of insulin corresponds to the insulin dose calculated on the basis of the blood glucose level measured by the blood glucose level measurement device 20. However, the present invention is not limited to this.

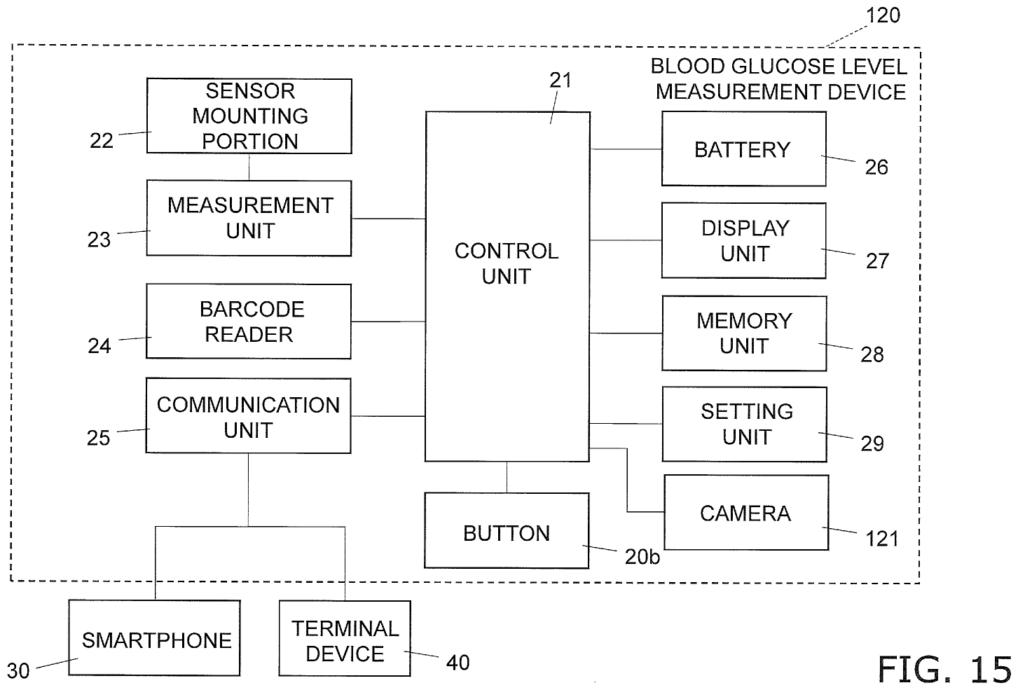

For example, if the blood glucose level measurement device is equipped with a camera function, as shown in FIG. 15, a camera (imaging unit) 121 installed in a blood glucose level measurement device (biometric information measurement device) 120 may be used to capture a confirmation image, and this image may be transmitted to the terminal device on the administrator side.

Alternatively, the communication terminal that captures the confirmation image may be a tablet terminal, a mobile phone, a PC with a camera, or the like, rather than a smartphone.

(D)

In the above embodiment, an example was given in which an image including a graduated portion of a pharmaceutical injection device such as a syringe containing insulin that will actually be administered is used as a confirmation image. However, the present invention is not limited to this.

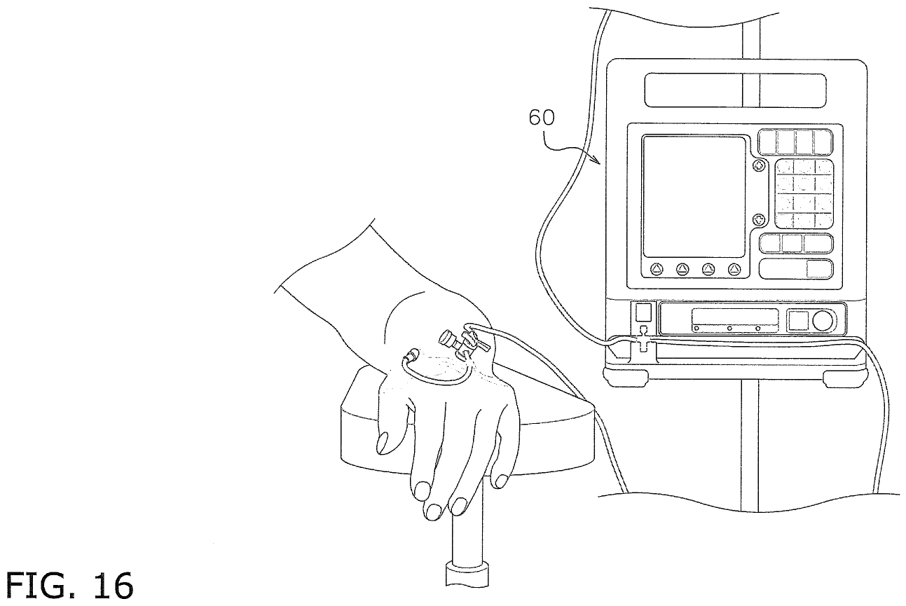

For example, as long as the information shows the amount of insulin that will actually be administered, a confirmation image including a numerical value that has been set for a pharmaceutical infusion pump 60 (including intravenous drip) including the insulin pump shown in FIG. 16 may be used.

Also, the confirmation image may include, for example, patient information such as the patient's face, patient ID, etc., in addition to information indicating the actual insulin dose.

This makes it possible to appropriately determine whether or not the proper amount of insulin will be administered to a specific patient.

(E)

In the above embodiment, an example was given in which the display 45 of the terminal device (PC or the like) 40 on the administrator side displayed text information about the insulin dose, patient ID, dosing person ID, and so forth along with confirmation images. However, the present invention is not limited to this.

For example, in the terminal device on the administrator side, only the confirmation images may be displayed to determine the suitability of the insulin dose, etc.

(F)

In the above embodiment, an example was given in which, when a confirmation image is transmitted from the smartphone 30 on the dosing person side to the terminal device 40 on the administrator side, the terminal device 40 outputs voice information and flashes a light to prompt the administrator to determine the suitability of the dose, etc., right away. However, the present invention is not limited to this.

For example, in the terminal device on the administrator side, the method of notifying the administrator by outputting voice information or light is not limited to the use of voice information or light, and may instead be some other method, such as applying vibration.

(G)

In the above embodiment, an example was given in which a PC or a tablet terminal was used as the terminal device 40 on the administrator side. However, the present invention is not limited to this.

For example, a biometric information measurement device such as a smartphone, a mobile phone, or a blood glucose level measurement device belonging to the administrator may be used as the terminal device on the administrator side.

(H)

In the above embodiment, an example was given in which the blood glucose level measurement device 20 was used as the biometric information measurement device, the blood glucose level was used as the biometric information, and insulin was used as the specific pharmaceutical. However, the present invention is not limited to this.

For example, as long as the type and dose of a specific pharmaceutical are set according to the biometric information measurement result, a combination of blood glucose level and insulin is not the only option, and a combination of some other biometric information and the specific pharmaceutical may be used.

(I)

In the above embodiment, an example was given in which a plurality of nurses who working in the same medical department (floor) of the hospital are registered as dosing persons and administrators in the terminal device 40 on the administrator side, and a combination of a dosing person and an administrator is set by referring to work shifts and so on. However, the present invention is not limited to this.

For example, the dosing person and the administrator may be registered in advance as fixed human resources.

Alternatively, a combination of dosing person and administrator is not limited to the work shift, and may instead be set by taking into account the experience, qualifications, specialties, etc. of the employees.

Also, the setting unit for setting the combination of a dosing person and an administrator does not need to be provided to the terminal device on the administrator side, and may, for example, be provided to a smartphone, a blood glucose level measurement device, or another such biometric information measurement device belonging to the dosing person, or to a server device or the like to which a terminal on the administrator side is connected.

Furthermore, the combination of the dosing person and the administrator may, for example, be set manually by the dosing person or the administrator, with the names, IDs, and so forth of a plurality of pre-registered employees displayed as a pull-down on the setting screen of the blood glucose level measurement device.

(J)

In the above embodiment, an example was given in which, when the administrator determines that the administration of insulin to the patient is NG after referring to the confirmation images received by the terminal device 40, the blood glucose level measurement result, the insulin calculation result, and the like, an option to re-measure the blood glucose level or re-capture a confirmation image is given to the dosing person side (the blood glucose level measurement device 20). However, the present invention is not limited to this.

For example, in addition to the above two options, if the insulin calculation result based on the blood glucose level measurement result is unsuitable, insulin recalculation may be added as an option.

Alternatively, the configuration may be such that only one of the above three options is presented to the dosing person.

(K)

In the above embodiment, an example was given in which a dedicated device for measuring blood glucose level was used as the blood glucose level measurement device 20. However, the present invention is not limited to this.

For example, a smartphone or the like equipped with a blood glucose level measurement function may be used as a biometric information measurement device.

(L)

In the above embodiment, an example was given in which the memory unit 28 that stored a list of nurses or the like corresponding to dosing persons and administrators was disposed in the blood glucose level measurement device 20. However, the present invention is not limited to this.

For example, a smartphone capable of communicating with the blood glucose level measurement device, a terminal device on the administrator side, or a memory unit provided to an external server or the like may be used.

Regardless of where the memory unit is located, combinations of dosing person and administrator can be set by referring to a list of nurses or the like corresponding to dosing persons and administrators via communication with a blood glucose level measurement device or other such biometric information measurement device.

### INDUSTRIAL APPLICABILITY

The pharmaceutical injection assessment system of the present invention has the effect of prompting an administrator to confirm the type and dose of a specific pharmaceutical set on the basis of a biometric information measurement result, and allowing the specific pharmaceutical to be reliably administered as dictated by the biometric information measurement result, and therefore can be widely applied to systems in which the type and dose of a specific pharmaceutical are set according to the measurement of biometric information.

### REFERENCE SIGNS LIST

1 sensor
1a deposition port
1b electrode portion
10 pharmaceutical injection assessment system
20 blood glucose level measurement device (biometric information measurement device)
20a main body unit
20b button
21 control unit
22 sensor insertion slot (sensor mounting portion)
23 measurement unit
24 barcode reader (information reading unit)
25 communication unit (pharmaceutical dose acquisition unit, first communication unit)
26 battery
27 display unit
28 memory unit
29 setting unit
30 smartphone (dosing person side)
31 control unit
32 camera (imaging unit)
33 input unit
34 communication unit
35 battery
36 display unit
37 memory unit
40 terminal device (administrator side)
41 control unit
42 keyboard (input unit)
43 mouse (input unit)
44 communication unit (reception unit, second communication unit)
45 display (display unit)
46 memory unit
47 output unit
50a, 50b confirmation image
51 syringe
51a graduated portion
60 pharmaceutical injection pump
120 blood glucose level measurement device (biometric information measurement device)
121 camera (imaging unit)
b1 blood (biological sample)
d1 insulin (specific pharmaceutical)
F1 finger

1. A pharmaceutical injection assessment system with which an administrator performs confirmation work related to an administration of a specific pharmaceutical on the basis of a measurement result for biometric information measured by a dosing person or a patient, the system comprising:
a biometric information measurement device configured to measure biometric information;
a pharmaceutical dose acquisition unit configured to acquire a type and dose of the specific pharmaceutical on the basis of the biometric information measured by the biometric information measuring device; and
a terminal device that has a receiving unit configured to receive information related to the type and dose of the specific pharmaceutical actually administered to the patient, and a display unit configured to display a display prompting the administrator to confirm the type and dose of the specific pharmaceutical.

2. The pharmaceutical injection assessment system according to claim 1,
further comprising an imaging unit configured to capture a confirmation image showing a type and dose of a specific pharmaceutical actually administered to the patient.

3. The pharmaceutical injection assessment system according to claim 2,
wherein the confirmation image includes a graduated portion showing the dose of the specific pharmaceutical in a state in which the specific pharmaceutical has been placed in a pharmaceutical injection device configured to administer the specific pharmaceutical to a patient.

4. The pharmaceutical injection assessment system according to claim 2,
wherein the confirmation image includes information about the patient to whom the specific pharmaceutical is to be administered.

5. The pharmaceutical injection assessment system according to claim 1,
wherein the biometric information measurement device has a measurement unit configured to measure the biometric information, and a first communication unit configured to transmit the measurement result from the measurement unit.

6. The pharmaceutical injection assessment system according to claim 2,
wherein the biometric information measurement device further has the pharmaceutical dose acquisition unit and the imaging unit.

7. The pharmaceutical injection assessment system according to claim 5,
further comprising an imaging unit for capturing a confirmation image showing the type and dose of the specific pharmaceutical that will actually be administered to the patient,
wherein the first communication unit transmits the confirmation image captured by the imaging unit.

8. The pharmaceutical injection assessment system according to claim 1,
wherein the pharmaceutical dose acquisition unit calculates the dose of a specific pharmaceutical on the basis of the biometric information measured by the biometric information measurement device.

9. The pharmaceutical injection assessment system according to claim 5,
wherein the pharmaceutical dose acquisition unit receives the type and dose of the specific pharmaceutical calculated on the basis of the biometric information transmitted from the first communication unit of the biometric information measurement device.

10. The pharmaceutical injection assessment system according to claim 1,
wherein the terminal device causes the display unit to display at least one set of text information from among the measurement result for biometric information, the type and dose of the specific pharmaceutical, a patient ID, and a dosing person ID.

11. The pharmaceutical injection assessment system according to claim 1,
wherein the terminal device further has an output unit configured to output voice information or light that prompts the administrator to make a determination regarding the administration of the specific pharmaceutical.

12. The pharmaceutical injection assessment system according to claim 1,
wherein the terminal device further has an input unit to which a result of determining a suitability of the type and dose of the specific pharmaceutical is inputted by the administrator who has confirmed the type and dose of the specific pharmaceutical.

13. The pharmaceutical injection assessment system according to claim 2,
wherein the terminal device further has a second communication unit configured to send the dosing person a message prompting one of the following: a result of a suitability determination made by the administrator who confirmed the type and dose of the specific pharmaceutical, a retake of the confirmation image, a re-measurement of the biometric information, and a recalculation of the dose of the specific pharmaceutical, or a message indicating that the specific pharmaceutical cannot be administered.

14. The pharmaceutical injection assessment system according to claim 1,
further comprising a memory unit for storing information about employees corresponding to the administrator and the dosing person.

15. The pharmaceutical injection assessment system according to claim 14,
wherein the memory unit further stores a work shift for the employees.

16. The pharmaceutical injection assessment system according to claim 15,
further comprising a setting unit for setting a combination of the dosing person and the administrator on the basis of the work shift for the employees stored in the memory unit.

17. The pharmaceutical injection assessment system according to claim 16,
wherein the combination of the dosing person and the administrator set in the setting unit is shared between the biometric information measurement device and the terminal device.

18. The pharmaceutical injection assessment system according to claim 14,
wherein the biometric information measurement device further has an information reading unit for reading a patient's personal information, and
the memory unit stores a setting range related to the type and dose of the specific pharmaceutical corresponding to the patient's personal information read by the information reading unit.

19. The pharmaceutical injection assessment system according to claim 1,
wherein the biometric information measurement device measures blood glucose level as the biometric information, and
the pharmaceutical dose acquisition unit acquires a dose of insulin as the specific pharmaceutical.

20. The pharmaceutical injection assessment system according to claim 2,
wherein the imaging unit is provided to a smartphone, a mobile phone, or a PC belonging to the dosing person.

21. A pharmaceutical injection assessment program with which an administrator performs confirmation work related to an administration of a specific pharmaceutical on the basis of a biometric information measurement result measured by a dosing person or a patient, wherein the program causes a computer to execute a pharmaceutical injection determination method comprising the steps of:
measuring the biometric information;
acquiring a type and dose of the specific pharmaceutical on the basis of the measured biometric information; and
receiving information about the type and dose of the specific pharmaceutical actually administered to the patient and displaying a display to prompt the administrator to confirm the type and dose of the specific pharmaceutical.
